(19) United States
(12) Patent Application Publication
(10) Pub. No.: US 2024/0415676 A1
(43) Pub. Date: Dec. 19, 2024

(54) SYSTEM AND METHOD FOR OPERATING A ROBOTIC PROSTHETIC LIMB

(71) Applicant: Alexandra Sepulveda, Holbrook, NY (US)

(72) Inventor: Alexandra Sepulveda, Holbrook, NY (US)

(21) Appl. No.: 18/210,200

(22) Filed: Jun. 15, 2023

(51) Int. Cl. A61F 2/72; B25J 9/00; B25J 9/16; G06F 3/01

(52) U.S. Cl. CPC A61F 2/72; B25J 9/0006; B25J 9/161; B25J 9/163; G06F 3/015; A61F 2002/6827; A61F 2002/704

(57) ABSTRACT

A system for operating a robotic prosthetic limb includes a brain machine interface (BMI) configured to sense neuron activity of a user and generate a signal indicating the sensed neuron activity and a robotic prosthetic limb configured to wirelessly communicate with the BMI. The robotic prosthetic limb includes motors connected configured to actuate the robotic prosthetic limb, a processor, and a memory. The memory includes instructions which when executed by the processor cause the system to: communicate a signal from the BMI to the robotic prosthetic limb, the signal including neuron activity of the user; input the neuron activity into four machine learning models configured to generate individual predictions of a movement of the robotic prosthetic limb; generate a motor control signal, for each of the one or more motors by averaging the individual predictions; and actuate the robotic prosthetic limb in response to the generated motor control signals.

### FIELD OF THE INVENTION

The present disclosure relates generally to prosthetic limb technologies and, more particularly, to wireless prosthetic limbs operated by a user's neurological system.

### BACKGROUND

A prosthesis, or a prosthetic implant, is used to replace a missing body appendage. An individual (an “amputee”) may have missing body appendages for a variety of reasons, including congenital disorders, trauma, or disease. Prosthetic limb technologies enable disabled persons to restore normal function to their missing body parts.

Present prosthetic limb technologies may utilize signals of the user's neurological system, such as neural signals sent from the brain. Such technologies generally employ predictive technologies to properly decipher and execute the intended motions of the prosthetic limb. However, current technologies are generally limited to using a single algorithm, frequently leading to delayed, inaccurate results. Moreover, current technologies can be cumbersome and dangerous to implant due to the delicate nature of the human neurological system.

Accordingly, there exists a need for improvements in wireless prosthetic limb technologies.

### SUMMARY

In accordance with aspects of the present disclosure, a system for operating a robotic prosthetic limb includes a brain machine interface (BMI) and a robotic prosthetic limb. The BMI is configured to generate a digitized signal and includes a plurality of electrodes, a processor, and a first wireless transceiver. The plurality of electrodes are configured to read a neural signal generated by the user. The processor is configured to digitize the neural signal and the first wireless transceiver transmits the digitized signal. The robotic prosthetic limb interacts with the BMI and includes a power source, a motor, a second wireless transceiver, and a controller. The motor is configured to actuate the robotic prosthetic limb. The second wireless transceiver is configured to receive a digitized signal from the first wireless transceiver. The controller executes a program having a set of four deep learning algorithms configured to generate a direction and speed prediction for the prosthetic limb.

In an aspect of the present disclosure, the BMI may include a plurality of electrodes may be configured to be implanted into the brain of a user and configured to read one or more neural signals generated by the user; a processor configured to digitize the neural signal read by the plurality of electrodes; and a first wireless transceiver configured to wirelessly communicate the digitized signal with the robotic prosthetic limb.

In another aspect of the present disclosure, the plurality of electrodes may include a flexible, biocompatible coating.

In yet another aspect of the present disclosure, the flexible, biocompatible coating may include silicone.

In a further aspect of the present disclosure, the robotic prosthetic limb may further include a second wireless transceiver configured to wirelessly communicate with the first wireless transceiver.

In an aspect of the present disclosure, the robotic prosthetic limb may further include a sensor configured to sense the movement of the robotic prosthetic limb, and wherein the instructions when executed by the processor further cause the system to: access the sensor signal indicating the movement; and compare the sensed movement to the predicted movement.

In another aspect of the present disclosure, the instructions when executed by the processor may further cause the system to: generate a sensory feedback signal, the sensory feedback signal configured to provide training data for the machine learning models; and train the machine learning models based on the sensory feedback signal.

In yet another aspect of the present disclosure, generating the prediction of the movement may include generating a direction and speed prediction for the prosthetic limb by each of the four machine learning models and averaging each of the direction predictions and the speed predictions of the four machine learning models to generate the motor control signal.

In a further aspect of the present disclosure, at least one machine learning model of the four machine learning models may be a convolutional neural network.

In an aspect of the present disclosure, the four machine learning models may use unsupervised learning.

In accordance with aspects of the present disclosure, a processor-implemented method for operating a robotic prosthetic limb is presented. The method includes communicating a signal from a brain machine interface (BMI) to the robotic prosthetic limb, the signal including neuron activity of a user, the (BMI) configured to sense neuron activity of the user and generate the signal indicating the sensed neuron activity; inputting the neuron activity into four machine learning models configured to generate individual predictions of a movement of a robotic prosthetic limb, the robotic prosthetic limb configured to wirelessly communicate with the BMI, wherein the robotic prosthetic limb includes one or more motors connected configured to actuate the robotic prosthetic limb; generating a motor control signal, for each of the one or more motors by averaging the individual predictions; and actuating the robotic prosthetic limb in response to the generated motor control signals.

In another aspect of the present disclosure, the BMI includes a plurality of electrodes may be configured to be implanted into the brain of a user and configured to read one or more neural signals generated by the user; a processor configured to digitize the neural signal read by the plurality of electrodes; and a first wireless transceiver configured to wirelessly communicate the digitized signal with the robotic prosthetic limb.

In yet another aspect of the present disclosure, the plurality of electrodes may include a flexible, biocompatible coating.

In a further aspect of the present disclosure, the flexible, biocompatible coating may include silicone.

In an aspect of the present disclosure, the robotic prosthetic limb may further include a second wireless transceiver configured to wirelessly communicate with the first wireless transceiver.

In another aspect of the present disclosure, the robotic prosthetic limb may further include a sensor configured to sense the movement of the robotic prosthetic limb. The method may further include accessing the sensor signal indicating the movement; and comparing the sensed movement to the predicted movement.

In yet another aspect of the present disclosure, the method may further include generating a sensory feedback signal, the sensory feedback signal configured to provide training data for the machine learning models and training the machine learning models based on the sensory feedback signal.

In a further aspect of the present disclosure, generating the prediction of the movement may include generating a direction and speed prediction for the prosthetic limb by each of the four machine learning models and averaging each of the direction predictions and the speed predictions of the four machine learning models to generate the motor control signal.

In an aspect of the present disclosure, at least one machine learning model of the four machine learning models may be a convolutional neural network.

In accordance with aspects of the present disclosure, a non-transitory computer readable medium storing a processor-implemented method for operating a robotic prosthetic limb, the method comprising: communicating a signal from a brain machine interface (BMI) to the robotic prosthetic limb, the signal including neuron activity of a user, the (BMI) configured to sense neuron activity of the user and generate a signal indicating the sensed neuron activity; inputting the neuron activity into four machine learning models configured to generate individual predictions of a movement of a robotic prosthetic limb, the robotic prosthetic limb configured to wirelessly communicate with the BMI, wherein the robotic prosthetic limb includes one or more motors connected configured to actuate the robotic prosthetic limb; generating a motor control signal, for each of the one or more motors by averaging the individual predictions; and actuating the robotic prosthetic limb in response to the generated motor control signals.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying drawings of which:

FIG. 1 is a diagram of a system for wirelessly operating a robotic prosthetic limb, in accordance with aspects of the disclosure;

FIG. 2 is a diagram of a brain machine interface (BMI) of with the system of FIG. 1, in accordance with aspects of the disclosure;

FIG. 3A is a flow diagram of the BMI for use with the system of FIG. 1, in accordance with aspects of the disclosure;

FIG. 3B is a flow diagram of the prosthetic robotic limb brain of the system of FIG. 1, in accordance with aspects of the disclosure;

FIGS. 4A and 4B are flow diagrams illustrating exemplary flow diagrams of the system of FIG. 1, in accordance with aspects of the disclosure;

FIG. 5 is a flow diagram illustrating feedback training for use with the system of FIG. 1, in accordance with aspects of the disclosure;

FIG. 6 is a block diagram of a controller configured for use with the system of FIG. 1, in accordance with aspects of the disclosure;

FIG. 7 is a block diagram of a machine learning network with inputs and outputs of a deep learning neural network, in accordance with aspects of the disclosure;

FIG. 8 is a diagram of layers of the machine learning network of FIG. 7, in accordance with aspects of the disclosure; and

FIG. 9 is a flow diagram of an exemplary method for wirelessly controlling the system of FIG. 1, in accordance with aspects of the disclosure.

### DETAILED DESCRIPTION

Aspects of the present disclosure are described in detail with reference to the drawings wherein like reference numerals identify similar or identical elements.

The phrases “in an aspect,” “in aspects,” “in various aspects,” “in some aspects,” or “in other aspects” may each refer to one or more of the same or different aspects in accordance with the present disclosure.

Although the present disclosure will be described in terms of specific aspects, it will be readily apparent to those skilled in this art that various modifications, rearrangements, and substitutions may be made without departing from the spirit of the present disclosure. The scope of the present disclosure is defined by the claims appended hereto. For purposes of promoting an understanding of the principles of the present disclosure, reference will now be made to exemplary aspects illustrated in the drawings, and specific language will be used to describe the same.

The present disclosure includes a system for operating a robotic prosthetic limb having a brain machine interface (BMI) and a wireless prosthetic limb.

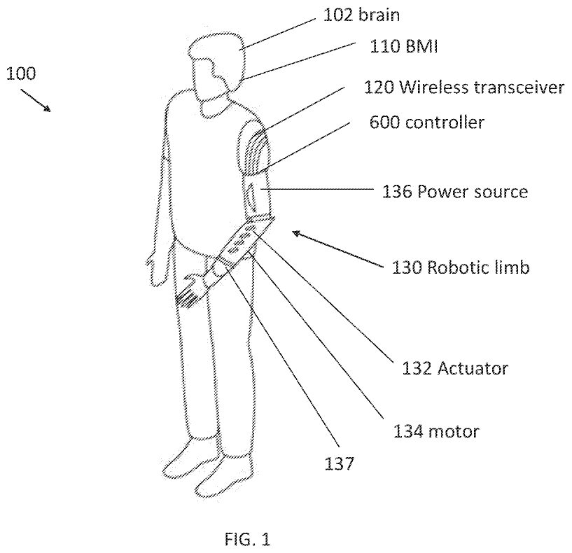

With reference to FIG. 1, a system 100 for operating a robotic prosthetic limb 130 is shown. The system 100 generally includes a brain machine interface (BMI) 200 in wireless communication with a robotic prosthetic limb 130. BMI 200 may be implanted in a user's neurological system, such as their brain 102. Robotic prosthetic limb 130 may be used to replace various body parts of a user. For example, robotic prosthetic limb 130 may be employed to replace an arm and/or a leg. It is contemplated that multiple robotic prosthetic limbs 130 may be used, for example, both legs, or an arm and a leg. The disclosed system 100 provides the benefit of enabling the operation of multiple robotic prosthetic limbs 130 using the same BMI 200. The BMI 200 may be configured to be a bi-directional BMI, configured for communicating signals not only from the brain but to the brain as well.

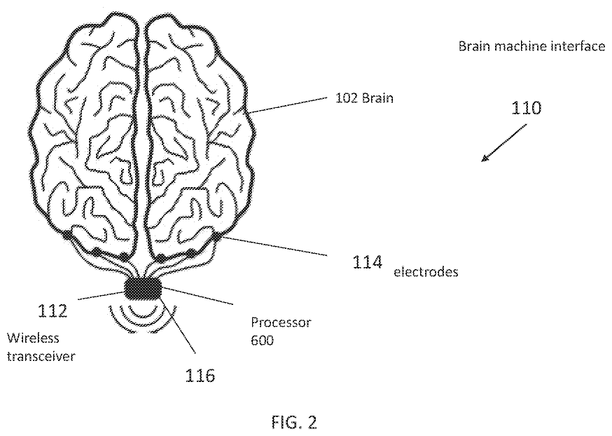

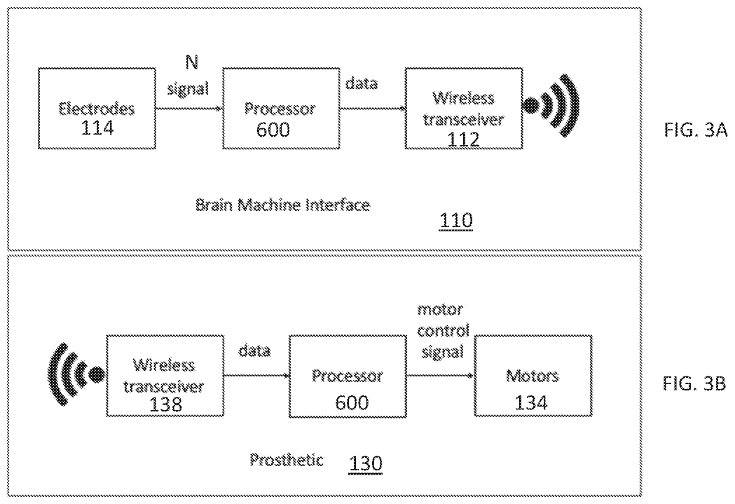

With reference to FIGS. 2 and 3A, BMI 200 for use with system 100 is shown. BMI 200 is configured to generate a digitized signal for robotic prosthetic limb 130. Generally, BMI 200 includes electrodes 114, controller 600, and wireless transceiver 230. Electrodes 114 are implanted into the brain of the user and configured to read a neural signal N generated by the user. For example, the user may attempt to move a joint related to their missing arm, causing their brain to generate a neural signal for generating arm movement, which may be read by the electrodes. In aspects, electrodes 114 may be coated with a flexible, biocompatible material to reduce damage to tissue of the user's brain.

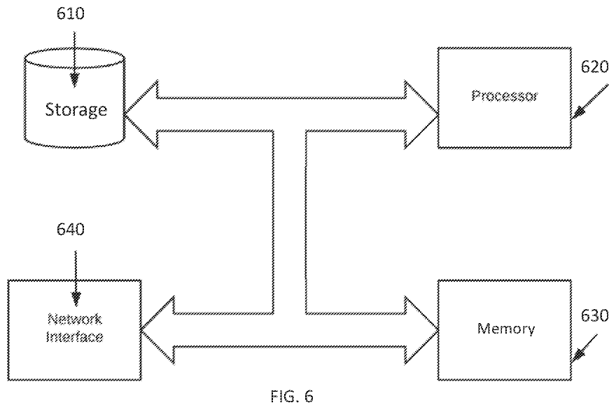

Controller 600 is configured to digitize the neural signal read by electrodes 114 (FIG. 6). For example, controller 600 may receive the neural signal for generating arm movement, consisting of electrical impulses. Controller 600 may execute an algorithm to convert this neural signal into computer-readable instructions, creating a digitized signal D. Digitized signal D may contain data including instructions for the motion of robotic prosthetic limb 130. Wireless transceiver 112 is configured to transmit digitized signal D to robotic prosthetic limb 130.

In aspects, BMI 200 may contain a power source 116 for powering BMI 200. The power source may be a rechargeable battery, which is capable of being recharged by a wireless charger (not shown).

With further reference to FIGS. 1 and 3B, the robotic prosthetic limb 130 generally includes a wireless transceiver 138, one or more motors 134, one or more actuators 132, a controller 600 (FIG. 6), and a power source 136. One of skill in the art would be familiar with how the motors in a robotic prosthetic limb work to actuate movement in response to a motor control signal.

The wireless transceiver 138 is configured to communicate with the wireless transceiver 112 of the BMI 110 to communicate processed neurological activity as data to the controller 600 of the robotic prosthetic limb 130, which is converted to motor control signals. The wireless transceiver 138 provides the benefit of eliminating external wires for communication between the BMI 200 and the robotic prosthetic limb 130. The motors 134 and actuators 132 are configured to enable movement in the limb, for example, moving an elbow, moving individual fingers, and/or rotating a wrist. The motors 134 may be connected to power source 136. The motors 134 may include a stator, a commutator, and a rotor. The controller 600 is a physically separate controller from the one used in the BMI 110. The power source 136 is configured to power the controller 600, the wireless transceiver 138, the motors 134 and the actuators 132. Power source 310 may be a removable battery. In aspects, power source 136 may be a rechargeable battery, which is capable of being recharged by a wireless charger (not shown).

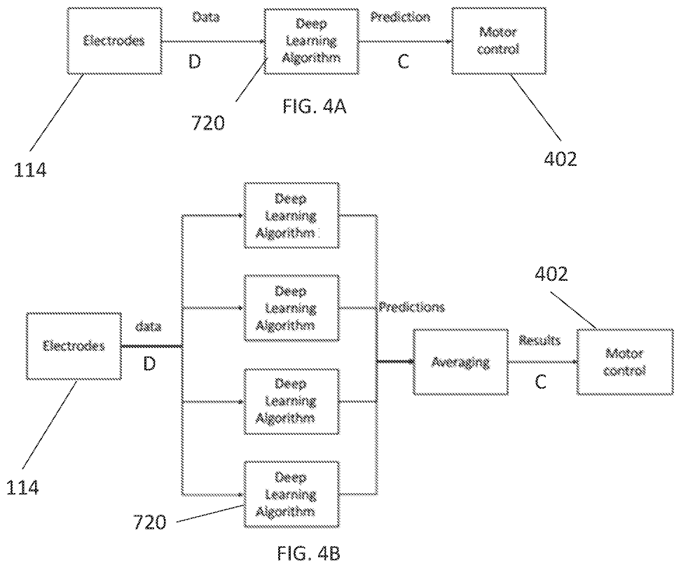

With further reference to FIGS. 3A and 3B, wireless transceiver 138 is configured to receive and/or transmit digitized signal D from wireless transceiver 112. For example, wireless transceiver 138 may receive a signal from wireless transceiver 112 containing programmatic instructions for the motion of robotic prosthetic limb 130 (e.g., movement of an arm). Controller 600 is configured to process digitized signal D using various deep learning algorithms (FIGS. 4A and 4B). After receiving digitized signal D, controller 600 may use a set of deep learning algorithms 720, for example, a set of four deep learning algorithms 720, to generate a predicted movement of the robotic prosthetic limb 130. Each deep learning algorithm 720 is different from one another, and each one is focused on a different aspect, such as speed, accuracy, size, or processing power. For example, each of each deep learning algorithms 720 could be a CNN, but they may have a different number of layers. Or in another example, there may be two CNNs with a different structure, e.g., a random forest and a support vector machine (SVM).

The disclosed technology provides a benefit over using predefined mapping of neurological activity to motor function, by using the machine learning to develop customized control for each patient. This solves the technological problem caused by predefined mapping of being inaccurate for a particular user. Additionally, by using machine learning, the disclosed technology provides at least the practical application of providing customized motor control signals for each robotic prosthetic limb of each user.

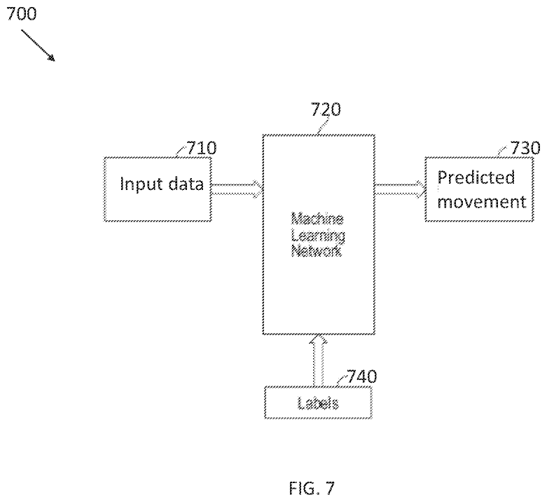

Each deep learning algorithm 720 is configured to process digitized signal D and generate a prediction 730 (FIG. 7). In aspects, the deep learning algorithm(s) 720 may include a convolutional neural network (CNN), although various types of deep learning algorithms are contemplated. Generally, the deep learning algorithms 720 are unsupervised algorithms, although supervised algorithms are also contemplated. Generally, prediction 520 includes a speed prediction and a direction prediction for the prosthetic limb, although various other predictions are contemplated. For example, prediction 730 may include a prediction that the user wants to move an arm at a 90-degree angle. Each of the deep learning algorithms 720 of the set of deep learning algorithms is configured to generate its own prediction, which is then averaged to a single control signal C for the motors 134 (FIG. 4B). This provides the technical benefit of a more accurate prediction at a faster speed, thus easier and more accurate movement of the user's robotic prosthetic limb 130. This also provides the technical benefit of using reduced processor power and saved energy in the robotic prosthetic limb's 130 power source 136, for example, enabling the use of smaller batteries.

It is contemplated that of the set of deep learning algorithms 720, some may use supervised training, and others may use unsupervised training. For example, in a case where four deep learning algorithms 720 are used, two may be pre-trained and use supervised training, and the other two may use unsupervised training.

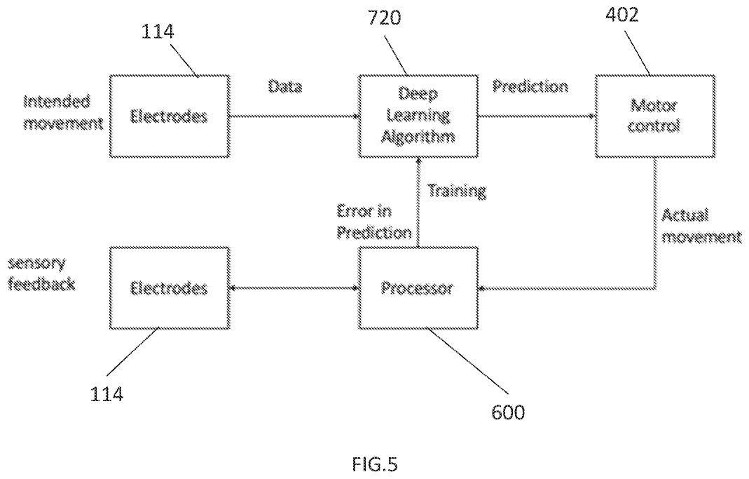

Referring to FIG. 5, a flow diagram of using sensory feedback to provide training data is shown. The robotic prosthetic limb 130 may further include one or more sensors 137 configured to sense the movement of the robotic prosthetic limb. Ten one or more sensors 137 may include accelerometers, gyroscopes, or any sensor capable of sensing movement.

After the robotic prosthetic limb 130 is actuated, the user may receive sensory feedback indicating the resulting direction and speed of the robotic prosthetic limb 130. For example, the user's initial neural signal may have indicated a desire to move their arm 90 degrees, however, the sensory feedback shows that the arm was only moved about 45 degrees. This sensory feedback may be fed back into controller 600 for use as training data for the deep learning algorithms 720.

For example, the controller 600 may cause the system 100 to access the sensor signal indicating the movement; compare the sensed movement to the predicted movement; generate a sensory feedback signal configured to provide training data for the machine learning models; and train the machine learning models based on the sensory feedback signal. In aspects, the controller 600 may determine somatosensory feedback through intracortical microstimulation (ICMS). ICMS provides feedback to the deep learning algorithms for training by stimulating the brain to provide tactile feedback in response to using the limb. It does this by using the electrodes of the BMI to stimulate portions of the brain that are responsible for sensory feedback. The use of ICMS enables the control of the robotic prosthetic limb 130 to be customized further. Customization improves the accuracy of the prosthetic as well as the user experience. This provides the benefit of improving ease of use for the user and increasing user adoption.

FIG. 6 illustrates controller 600, which includes processor 620 connected to a computer-readable storage medium or memory 630. The computer-readable storage medium or memory 630 may be a volatile type of memory, e.g., RAM, or a non-volatile type of memory, e.g., flash media, disk media, etc. In various aspects of the disclosure, the processor 620 may be another type of processor, such as a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 630 can be random access memory, read-only memory, magnetic disk memory, solid-state memory, optical disc memory, and/or another type of memory. In some aspects of the disclosure, the memory 630 can be separate from the controller 600 and can communicate with the processor 620 through communication buses of a circuit board and/or through communication cables such as serial cables or other types of cables. Memory 630 includes computer-readable instructions that are executable by the processor 620 to operate the controller 600. In other aspects of the disclosure, the controller 600 may include a network interface 640 to communicate with other computers or to a server. A storage device 610 may be used for storing data. The disclosed method may run on the controller 600 or on a user device, including, for example, on a mobile device, an IoT device, or a server system.

With reference to FIG. 7, a block diagram for a machine learning network 720 (e.g., a machine learning model) for classifying data in accordance with some aspects of the disclosure is shown. In some systems, a machine learning network 720 may include, for example, a convolutional neural network (CNN) and/or a recurrent neural network. A deep learning neural network includes multiple hidden layers. As explained in more detail below, the machine learning network 320 may leverage one or more classification models (e.g., CNNs, decision trees, Naive Bayes, k-nearest neighbor) to classify data. The machine learning network 720 may be executed on the controller 600 (FIG. 6). Persons of ordinary skill in the art will understand the machine learning network 720 and how to implement it.

In machine learning, a CNN is a class of artificial neural network (ANN). The convolutional aspect of a CNN relates to applying matrix processing operations to localized portions of the data, and the results of those operations (which can involve dozens of different parallel and serial calculations) are sets of many features that are delivered to the next layer. A CNN typically includes convolution layers, activation function layers, deconvolution layers (e.g., in segmentation networks), and/or pooling (typically max pooling) layers to reduce dimensionality without losing too many features. Additional information may be included in the operations that generate these features. Providing unique information that yields features that give the neural networks information can be used to provide an aggregate way to differentiate between different data being input to the neural networks.

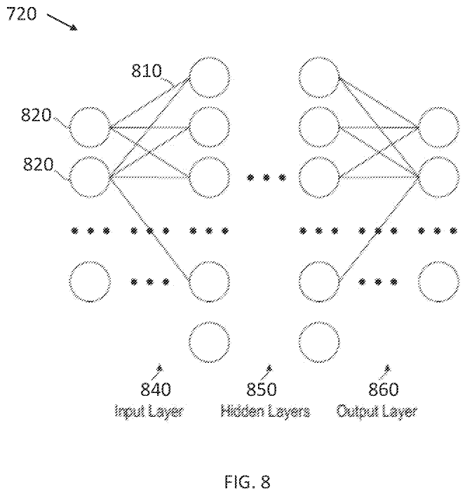

Referring to FIG. 8, generally, a machine learning network 720 (e.g., a convolutional deep learning neural network) includes at least one input layer 840, a plurality of hidden layers 850, and at least one output layer 860. The input layer 840, the plurality of hidden layers 850, and the output layer 860 all include neurons 820 (e.g., nodes). The neurons 820 between the various layers are interconnected via weights 810. Each neuron 820 in the machine learning network 720 computes an output value by applying a specific function to the input values coming from the previous layer. The function that is applied to the input values is determined by a vector of weights 810 and a bias. Learning in the deep learning neural network progresses by making iterative adjustments to these biases and weights. The vector of weights 810 and the bias are called filters (e.g., kernels) and represent particular features of the input (e.g., a particular shape). The machine learning network 720 may output logits. Although CNNs are used as an example, other machine learning classifiers are contemplated.

The machine learning network 720 may be initially trained based on feeding a dataset into the machine learning network 720 that is known to be good and/or ground truth performance data. This enables the machine learning network 720 to learn what good performance looks like and detect deviations through unsupervised learning.

The machine learning network 720 may undergo supervised learning. The machine learning network 720 may be trained based on labeled training data to optimize weights. For example, samples of sensor feature data may be taken and labeled using other sensor feature data. In some methods in accordance with this disclosure, the training may include supervised learning or semi-supervised. Persons of ordinary skill in the art will understand training the machine learning network 720 and how to implement it.

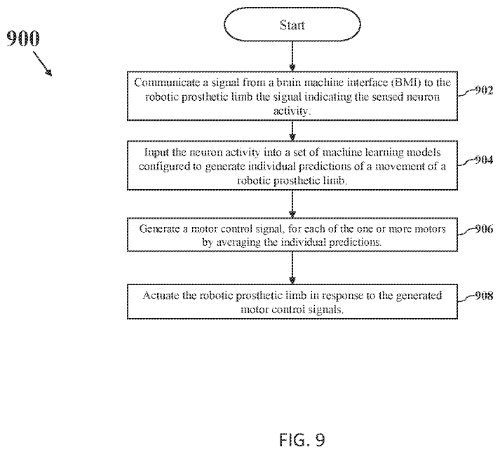

Referring to FIG. 9, a flow diagram for a method in accordance with the present disclosure for wireless robotic limb control is shown as 900. Although the steps of FIG. 9 are shown in a particular order, the steps need not all be performed in the specified order, and certain steps can be performed in another order. For example, FIG. 9 will be described below, with a controller 600 of FIG. 6, for example the controller in the robotic prosthetic limb 130, performing the operations. Some operations may be executed by the controller in the BMI 200. These variations are contemplated to be within the scope of the present disclosure.

At step 902, the controller 600 causes the system 100 to communicate a signal from the BMI 200 to the robotic prosthetic limb 130 (FIGS. 3A and 3B). The signal includes neurological activity of the user. When an amputee tries to move their amputated limb, the same neural signals that would normally happen in their brain are still present. For example, the user may intend to move a limb, such as the arm. Certain neurons in the user's brain may have activity. The plurality of electrodes 114 which are implanted into the brain of the user are configured to read the neural signals generated by the user. In order to not tear the brain tissue, the electrodes may be coated with a flexible biocompatible material such as silicon. The BMI 200 includes a processor 620 configured to digitize the neural signal read by the plurality of electrodes 114. The BMI 200 includes a first wireless transceiver 112 that wirelessly communicates the digitized signal with the robotic prosthetic limb 130.

At step 904, the controller 600 causes the system 100 to input the neuron activity into four machine learning models configured to generate individual predictions of a movement of the robotic prosthetic limb (FIG. 4B). The machine learning model would be primarily executed on the processor/controller of the robotic prosthetic limb 130. This is beneficial because machine learning models can cause high power consumption, thus requiring a larger battery/power source. This provides the improvement to technology of having a smaller controller/processor in the BMI 200, that needs to be charged less often due to the reduced current consumption.

In aspects, the controller 600 may select the most energy efficient machine learning model (e.g., deep learning algorithm 720) for each model that provides an accuracy within a predetermined amount. For example, the controller 600 may select an energy-based deep learning model, as one or more of the plurality of deep learning algorithms 720. The system 100 may include low power processors, or processors using low power modes, configured to leverage the reduced load of the energy-based deep learning models and save energy. The disclosed technology helps mitigate climate change by dynamically reducing the power required to operate and reducing a number of times the batteries of the system 100 would need to be charged, thus reducing land fill waste by lengthening battery life.

At step 906, the controller 600 causes the system 100 to generate a motor control signal for each of the one or more motors by averaging the individual predictions (FIG. 4B). In a simplified example, the first ML model may generate a prediction of moving an elbow of the robotic prosthetic limb 130 about 15 degrees at a velocity of about 1 meter per second, the second ML model may generate a prediction of moving the elbow of the robotic prosthetic limb 130 about 11 degrees at a velocity of about 1.3 meter per second, the third ML model may generate a prediction of moving the elbow of the robotic prosthetic limb 130 about 15 degrees at a velocity of about 2 meters per second, and the fourth ML model may generate a prediction of moving the elbow of the robotic prosthetic limb 130 about 17 degrees at a velocity of about 1 meter per second. The controller 600 may cause the system to generate an average value of 14.5 degrees at a velocity of 1.275 meters per second. These values are then converted to a motor control signal configured to cause the movement of the elbow of the robotic prosthetic limb 130.

At step 908, the controller 600 causes the system 100 to actuate the robotic prosthetic limb in response to the generated motor control signals (FIG. 4B).

It should be understood that the foregoing description is only illustrative of the present disclosure. Various alternatives, modifications, and variances can be devised by those skilled in the art without departing from the disclosure. For instance, although certain aspects herein are described as separate aspects, each of the aspects herein may be combined with one or more of the other aspects herein. Specific structural and functional details disclosed herein are not to be interpreted as limiting but as a basis for the claims and as a representative basis for teaching one skilled in the art to variously employ the present disclosure in any appropriately detailed structure. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for operating a robotic prosthetic limb, comprising:
a brain machine interface (BMI) configured to sense neuron activity of a user and generate a signal indicating the sensed neuron activity; and
a robotic prosthetic limb configured to wirelessly communicate with the BMI, the robotic prosthetic limb including:
one or more motors connected configured to actuate the robotic prosthetic limb;
a processor; and
a memory, including instructions stored thereon, which when executed by the processor cause the system to:
communicate a signal from the BMI to the robotic prosthetic limb, the signal including neuron activity of the user;
input the neuron activity into four machine learning models configured to generate individual predictions of a movement of the robotic prosthetic limb;
generate a motor control signal, for each of the one or more motors by averaging the individual predictions; and
actuate the robotic prosthetic limb in response to the generated motor control signals.

2. The system of claim 1, wherein the BMI includes:
a plurality of electrodes are configured to be implanted into the brain of a user and configured to read one or more neural signals generated by the user;
a processor configured to digitize the neural signal read by the plurality of electrodes; and
a first wireless transceiver configured to wirelessly communicate the digitized signal with the robotic prosthetic limb.

3. The system of claim 2, wherein the plurality of electrodes include a flexible, biocompatible coating.

4. The system of claim 3, wherein the flexible, biocompatible coating includes silicone.

5. The system of claim 2, wherein the robotic prosthetic limb further includes a second wireless transceiver configured to wirelessly communicate with the first wireless transceiver.

6. The system of claim 1, wherein the robotic prosthetic limb further includes a sensor configured to sense the movement of the robotic prosthetic limb, and wherein the instructions when executed by the processor further cause the system to:
access the sensor signal indicating the movement; and
compare the sensed movement to the predicted movement.

7. The system of claim 6, wherein the instructions when executed by the processor further cause the system to:
generate a sensory feedback signal, the sensory feedback signal configured to provide training data for the machine learning models; and
train the machine learning models based on the sensory feedback signal.

8. The system of claim 1, wherein generating the prediction of the movement includes:
generating a direction and speed prediction for the prosthetic limb by each of the four machine learning models; and
averaging each of the direction predictions and the speed predictions of the four machine learning models to generate the motor control signal.

9. The system of claim 1, wherein at least one machine learning model of the four machine learning models is a convolutional neural network.

10. The system of claim 1, wherein the four machine learning models use unsupervised learning.

11. A processor-implemented method for operating a robotic prosthetic limb, the method comprising:
communicating a signal from a brain machine interface (BMI) to the robotic prosthetic limb, the signal including neuron activity of a user, the (BMI) configured to sense neuron activity of the user and generate the signal indicating the sensed neuron activity;
inputting the neuron activity into four machine learning models configured to generate individual predictions of a movement of a robotic prosthetic limb, the robotic prosthetic limb configured to wirelessly communicate with the BMI, wherein the robotic prosthetic limb includes one or more motors connected configured to actuate the robotic prosthetic limb;
generating a motor control signal, for each of the one or more motors by averaging the individual predictions; and
actuating the robotic prosthetic limb in response to the generated motor control signals.

12. The processor-implemented method of claim 11, wherein the BMI includes:
a plurality of electrodes are configured to be implanted into the brain of a user and configured to read one or more neural signals generated by the user;
a processor configured to digitize the neural signal read by the plurality of electrodes; and
a first wireless transceiver configured to wirelessly communicate the digitized signal with the robotic prosthetic limb.

13. The processor-implemented method of claim 12, further comprising selecting each of the four machine learning models based on an energy used by each of the four machine learning models and further based on an accuracy of the four machine learning models being within a predetermined threshold.

14. The processor-implemented method of claim 13, wherein the plurality of electrodes include a flexible, biocompatible coating, and wherein the flexible, biocompatible coating includes silicone.

15. The processor-implemented method of claim 12, wherein the robotic prosthetic limb further includes a second wireless transceiver configured to wirelessly communicate with the first wireless transceiver.

16. The processor-implemented method of claim 11, wherein the robotic prosthetic limb further includes a sensor configured to sense the movement of the robotic prosthetic limb, and wherein the method further comprises:
accessing the sensor signal indicating the movement; and
comparing the sensed movement to the predicted movement.

17. The processor-implemented method of claim 16, further comprising:
generating a sensory feedback signal, the sensory feedback signal configured to provide training data for the machine learning models; and
training the machine learning models based on the sensory feedback signal.

18. The processor-implemented method of claim 11, wherein generating the prediction of the movement includes:
generating a direction and speed prediction for the prosthetic limb by each of the four machine learning models; and
averaging each of the direction predictions and the speed predictions of the four machine learning models to generate the motor control signal.

19. The processor-implemented method of claim 11, wherein at least one machine learning model of the four machine learning models is a convolutional neural network.

20. A non-transitory computer readable medium storing a processor-implemented method for operating a robotic prosthetic limb, the method comprising:
communicating a signal from a brain machine interface (BMI) to the robotic prosthetic limb, the signal including neuron activity of a user, the (BMI) configured to sense neuron activity of the user and generate a signal indicating the sensed neuron activity;
inputting the neuron activity into four machine learning models configured to generate individual predictions of a movement of a robotic prosthetic limb, the robotic prosthetic limb configured to wirelessly communicate with the BMI, wherein the robotic prosthetic limb includes one or more motors connected configured to actuate the robotic prosthetic limb;
generating a motor control signal, for each of the one or more motors by averaging the individual predictions; and
actuating the robotic prosthetic limb in response to the generated motor control signals.
